(19) United States
(12) Patent Application Publication
(10) Pub. No.: US 2021/0282910 A1
(43) Pub. Date: Sep. 16, 2021

(54) WORK LIGHT

(71) Applicant: Thomas Kerber, Hamilton (CA)

(72) Inventor: Thomas Kerber, Hamilton (CA)

(21) Appl. No.: 17/033,847

(22) Filed: Sep. 27, 2020

Related U.S. Application Data
(60) Provisional application No. 62/907,550, filed on Sep. 27, 2019.

(51) Int. Cl. A61C 17/06; F21V 17/02; F21V 23/06; F21V 19/00

(52) U.S. Cl. CPC A61C 17/084; F21V 17/02; F21Y 2115/10; F21V 19/003; F21V 23/06

(57) ABSTRACT

A work light comprises an elongate circuit board having a length extending between a connection end and a free end and a width extending between a left side and a right side. A light emitting diode is mounted on the elongate circuit board. An elongate protective cover has a length extending between a mounting end and a free end, and generally surrounds at least most of said elongate circuit board. The protective cover may comprise a suctioning tube having a fluid ingress and a fluid egress. The suctioning tube is connectable at the mounting end to a source of suction.

### FIELD OF THE INVENTION

The present invention relates to work lights, and more particularly to work lights that are inexpensive to manufacture and therefore readily disposable, and also to particularly to portable work lights with fluid suctioning past the source of light.

### BACKGROUND OF THE INVENTION

It is common in many fields of hands-on work to illuminate an overall area with one or more lights that illuminate an overall area from at least one angle. It is also necessary at times to have enclosed areas or internal areas illuminated. In this case, the work light would have a need for cooling.

Also, in the event that the work lights are used in applications such as medical applications, dental applications, research applications, experimental applications, veterinary applications, and so on, the work light might need to be either sterilized of be disposable, and therefore inexpensive to manufacture, or recyclable.

A prior art light device that combines both a source of illumination and a source of air suction is disclosed in United States Published Patent Application No. US2017/0231733A1, published Aug. 17, 2017, to Schmid et al, and entitled A Dental Light Device. In that document, a dental light device has a light source, specifically an LED mounted generally centrally between the front end and the back end of the dental light device, and arranged in thermal contact with a heat sink, for providing blue light and an air outlet arranged adjacent the light output. The air outlet is connected or connectable to an air supply. The dental light device provides for simultaneously irradiating an object and cooling the object.

Another prior art device used to illuminate the mouth of a patient is taught in United States Published Patent Application No. US2006/0269898A1, published Nov. 30, 2006, to Lefferson, and entitled Inside Mouth Dental Light. In that document, Lefferson discloses a dental lighting system comprising a light source enclosure having a length to cover one or more teeth of a patient and a width that is sufficient to allow placement on top of the teeth or gums of the patient. The light source enclosure is fastened inside patient's mouth over one or more teeth. A plurality of light sources, such as light emitting diodes, are mounted on the light source enclosure to shine inwardly into the mouth of the patient.

Yet another prior art device used to illuminate the mouth of a patient is taught in United States Published Patent Application No. US2007/0224571A1, published Sep. 27, 2007, to Watson, and entitled Light Mirror. One embodiment of the Watson invention provides a light device. The light device may include a mirror and comprises a handle having a longitudinal axis, a mirror attached to the handle and arranged at an angle from the longitudinal axis, a light source inside the handle, and a light waveguide adjacent to the light source. The waveguide comprises an internal reflector, and the reflector reflects substantially all of the light from the light waveguide. In one embodiment, an annular air flow substantially aligned with a longitudinal axis of the mirror cools the apparatus, including the light source. One embodiment of the light mirror keeps the reflective surface of the mirror free of debris, water, restorative materials, tooth structure, and aluminum oxide powder. Thus, the operator may continue to work without the constant cleaning of the mirror surface associated with conventional mirrors. Cleaning the mirror during a procedure can be time-consuming and counter-productive. Moreover, one embodiment of the light mirror is more ergonomic and reduces eye strain, enabling more accurate and precise results in far less time than possible with conventional light mirrors.

A light device for curing a dental composition is taught in United States Published Patent Application No. 2011/0236851, published Sep. 29, 2011, to Müller et al, and entitled Inside Mouth Dental Light. The disclose dental light comprises a light-source, a housing, a passive heat sink that is hermetically enclosed in the housing, first and second thermal pathways enabling heat transfer between the light source and the heat sink and between the heat sink and the housing. The first thermal pathway is dominated by thermal conduction, and the second thermal pathway is dominated by thermal radiation and thermal convection. This specific design provides an inexpensive and compact design for a dental light.

It is an object of the present invention to provide a work light that meets the needs as set forth above.

It is an object of the present invention to provide a work light that can readily illuminate an enclosed areas or internal area.

It is an object of the present invention to provide a work light that can readily illuminate the mouth of a patient.

It is an object of the present invention to provide a work light that has a low enough operating temperature to be comfortably used in the mouth of a patient.

It is an object of the present invention to provide a work light that is disposable.

It is an object of the present invention to provide a work light that is hygienic.

It is an object of the present invention to provide a work light that is inexpensive to manufacture.

It is an object of the present invention to provide a work light that is easy to use.

It is an object of the present invention to provide a work light that is bendable.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter of which is briefly described herein below.

### SUMMARY OF THE INVENTION

In accordance with one aspect of the present invention, there is disclosed a novel work light comprising an elongate circuit board having a length extending between a connection end and a free end and a width extending between a left side and a right side. A light emitting diode is mounted on the elongate circuit board. An elongate protective cover has a length extending between a mounting end and a free end, and generally surrounds at least most of the elongate circuit board.

In accordance with another aspect of the present invention, there is disclosed a novel work light comprising a circuit board and a light emitting diode mounted on the circuit board. A protective cover generally surrounding at least most of the circuit board. The protective cover comprises a suctioning tube having a fluid ingress and a fluid egress. The suctioning tube is connectable at the mounting end to a source of suction.

### BRIEF DESCRIPTION OF THE DRAWINGS

The novel features which are believed to be characteristic of the work light according to the present invention, as to its structure, organization, use and method of operation, together with further objectives and advantages thereof, will be better understood from the following drawings in which a presently preferred embodiment of the invention will now be illustrated by way of example. It is expressly understood, however, that the drawings are for the purpose of illustration and description only, and are not intended as a definition of the limits of the invention. In the accompanying drawings:

FIG. 1 is a perspective view from in front and above of the first illustrated embodiment work light according to the present invention;

FIG. 2 is a perspective view from behind and above of the first illustrated embodiment work light of FIG. 1;

FIG. 3 is a right side elevational view of the first illustrated embodiment work light of FIG. 1;

FIG. 4 is a left side elevational view of the first illustrated embodiment work light of FIG. 1;

FIG. 5 is a top plan view of the first illustrated embodiment work light of FIG. 1;

FIG. 6 is a bottom plan view of the first illustrated embodiment work light of FIG. 1;

FIG. 7 is a front end view of the first illustrated embodiment work light of FIG. 1;

FIG. 8 is a back end view of the first illustrated embodiment work light of FIG. 1;

FIG. 9 is a sectional right side elevational view of the first illustrated embodiment work light of FIG. 1, taken along section line 9-9 of FIG. 7;

FIG. 10 is a sectional top plan view of the first illustrated embodiment work light of FIG. 1, taken along section line 10-10 of FIG. 7;

FIG. 11 is a perspective view from behind and to the left of the first illustrated embodiment work light of FIG. 1, with the flexible circuit board and the elongate protective cover having been bent to a curved configuration for use;

FIG. 12 is a perspective view from above and to the left of the second illustrated embodiment work light according to the present invention;

FIG. 13 is an enlarged sectional right side elevational view of the first illustrated embodiment work light of FIG. 1, taken along section line 13-13 of FIG. 12;

FIG. 14 is a perspective view of the power control connector used in the first and second illustrated embodiments of the work light according to the present invention;

FIG. 15 is a perspective view of the closed end of the power control connector of FIG. 14;

FIG. 16 is an end perspective view of the open end of the power control connector of FIG. 14; and,

FIG. 17 is a perspective view of the work light, power control assembly and the source of DC power.

### LIST OF REFERENCE NUMERALS AND COMPONENTS

100 work light
101 front end
102 back end
120 elongate circuit board
“LB” length
“WB” width
120b back end portion
120p projecting end portion
121c connection end
121f free end
121l left side
121r right side
121t top surface
121b bottom surface
128 aluminum core
128a, 128b surfaces of the aluminum core
129c copper trace
129i electrically insulative material
130 light emitting diode
140 elongate protective cover
“LC” length
“WC” width
“HC” height
“D” diameter
141 mounting end
142 free end
143 left side
144 right side
145 top
146 bottom
147 throughpassage
148 fluid ingress
149 fluid egress
150 electrical connector assembly
150c electrical connector circuit
151 outer housing
152 input jack
154 edge connector
156 wires
170 main body
171 front end
172 back end
174 fluid-passing throughpassage
175 fluid ingress
176 fluid egress
177 circuit board throughpassage
180 power control assembly
182 power jack
184 USB connector
186 potentiometer or rheostat
188 cable/wire
190 source of DC power
191 USB cable
192 suctioning hose
193 connection end
200 work light
220 flexible circuit board
274 fluid-conducting throughpassage

### DETAILED DESCRIPTION OF THE ILLUSTRATED EMBODIMENTS

In accordance with one aspect of the present invention, there is disclosed a novel work light comprising an elongate circuit board having a length extending between a connection end and a free end and a width extending between a left side and a right side. A light emitting diode is mounted on the elongate circuit board. An elongate protective cover has a length extending between a mounting end and a free end, and generally surrounds at least most of the elongate circuit board.

In accordance with another aspect of the present invention, there is disclosed a novel work light comprising a circuit board and a light emitting diode mounted on the circuit board. A protective cover generally surrounding at least most of the circuit board. The protective cover comprises a suctioning tube having a fluid ingress and a fluid egress. The suctioning tube is connectable at the mounting end to a source of suction.

The present invention discloses a work light that is suitable for uses in various applications including, but not limited to medical applications, dental applications, research applications, experimental applications, veterinary applications, and so on. The illustrated comprises a dental light, specifically a dental light apparatus that is configured to suction saliva from a patient's mouth. As illustrated, the work light is inexpensive to manufacture, and therefore is recyclable or disposable. Other embodiments of the work light may be sterilized and reused.

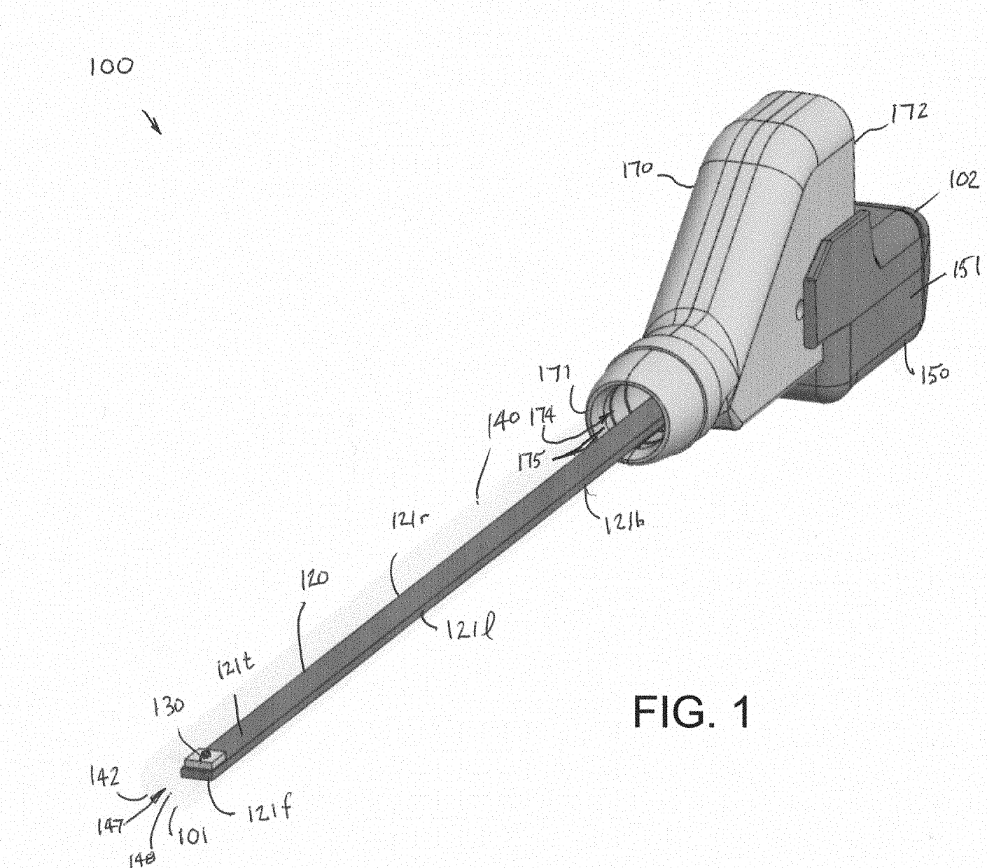

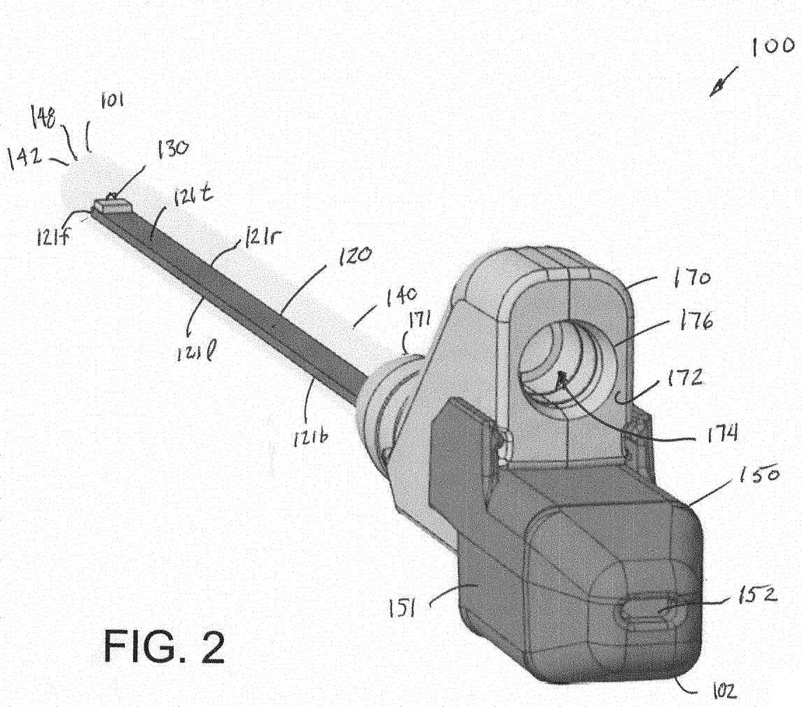

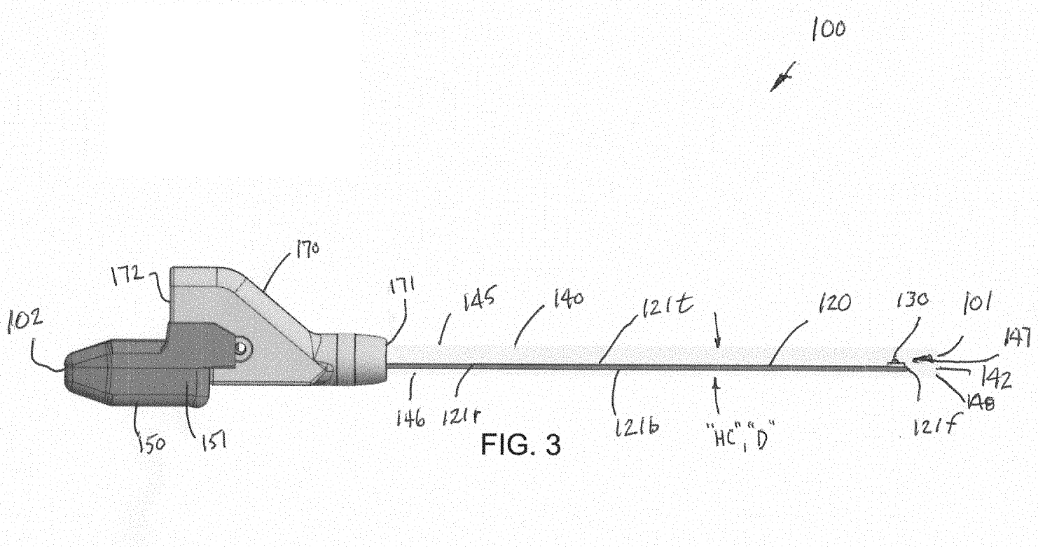

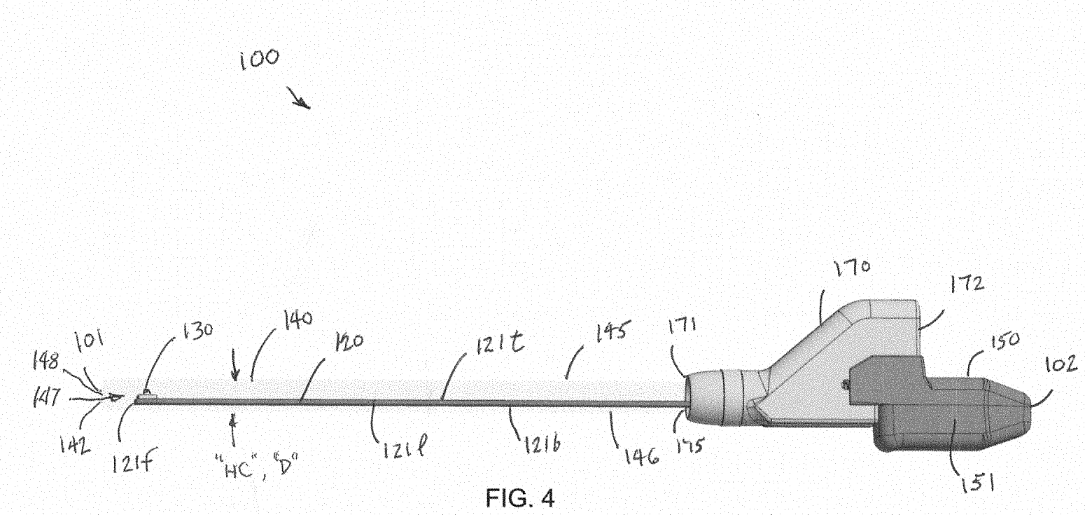

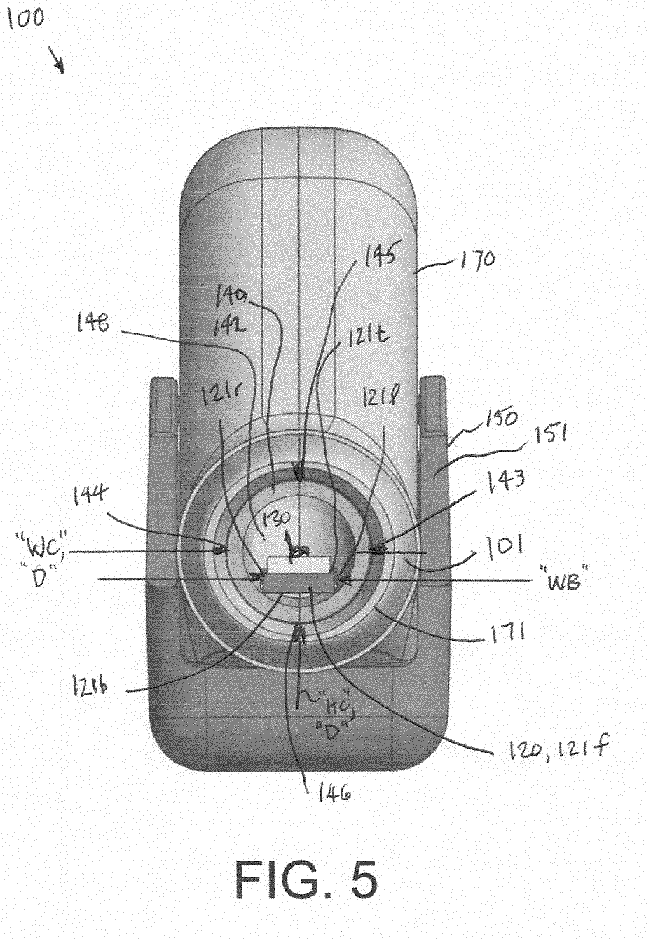

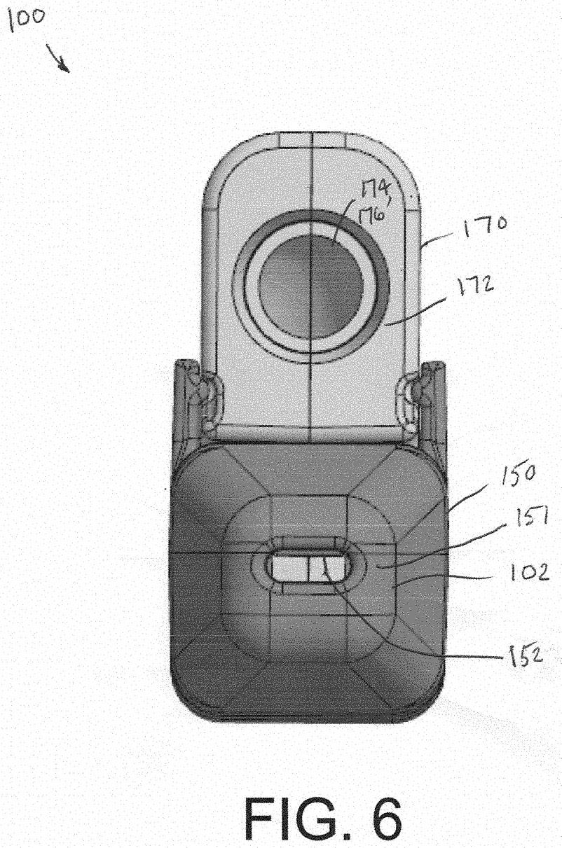

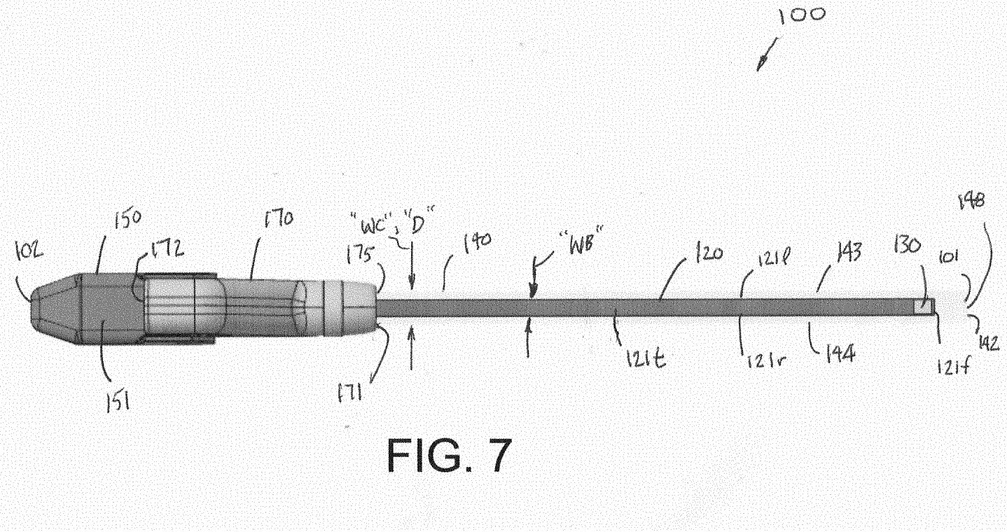

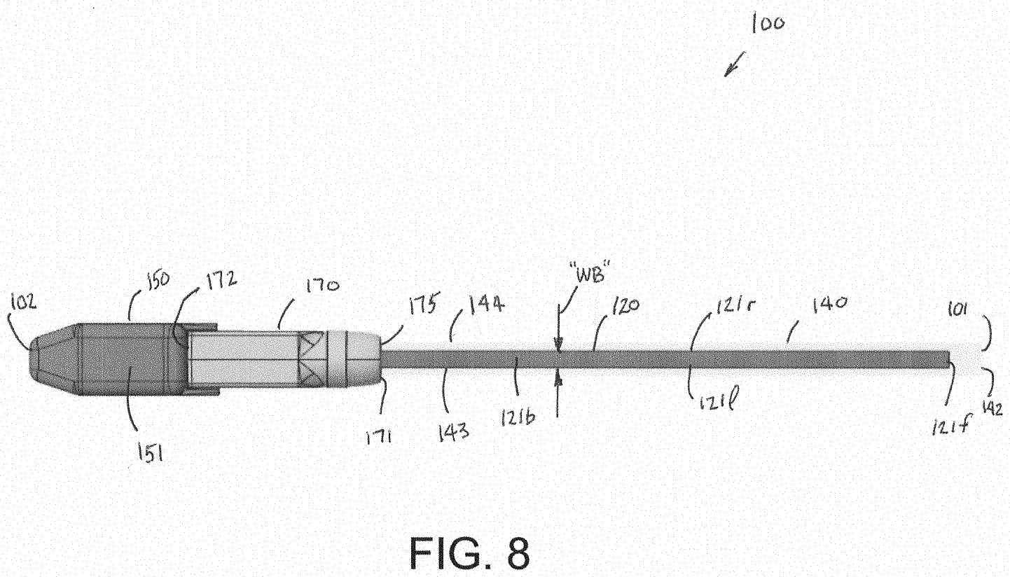

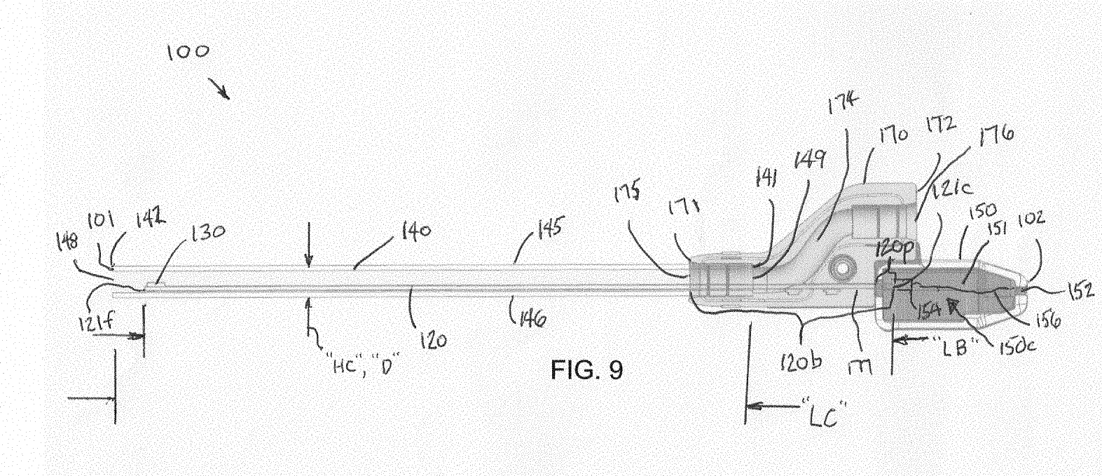

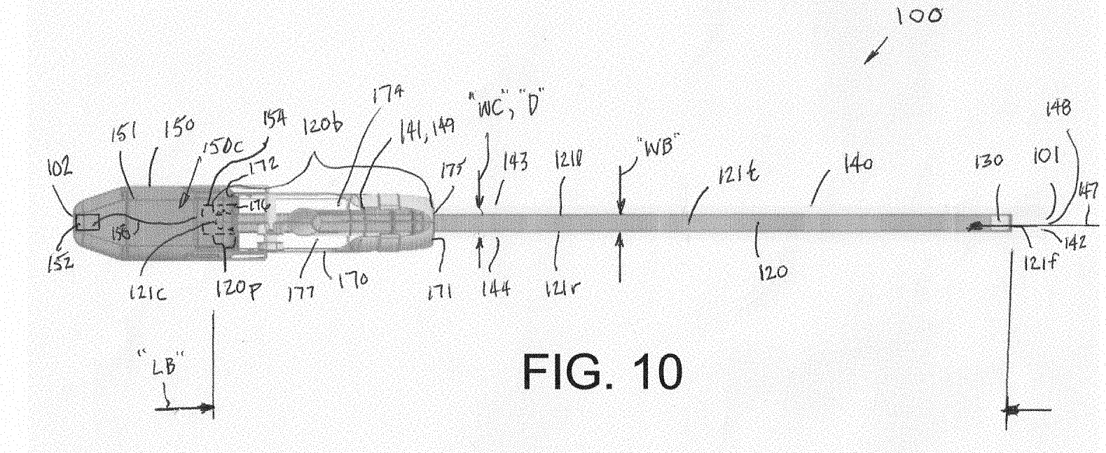

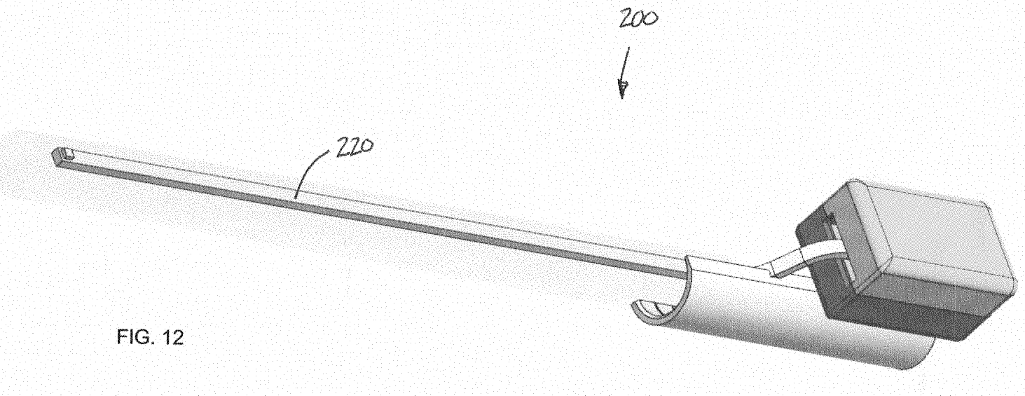

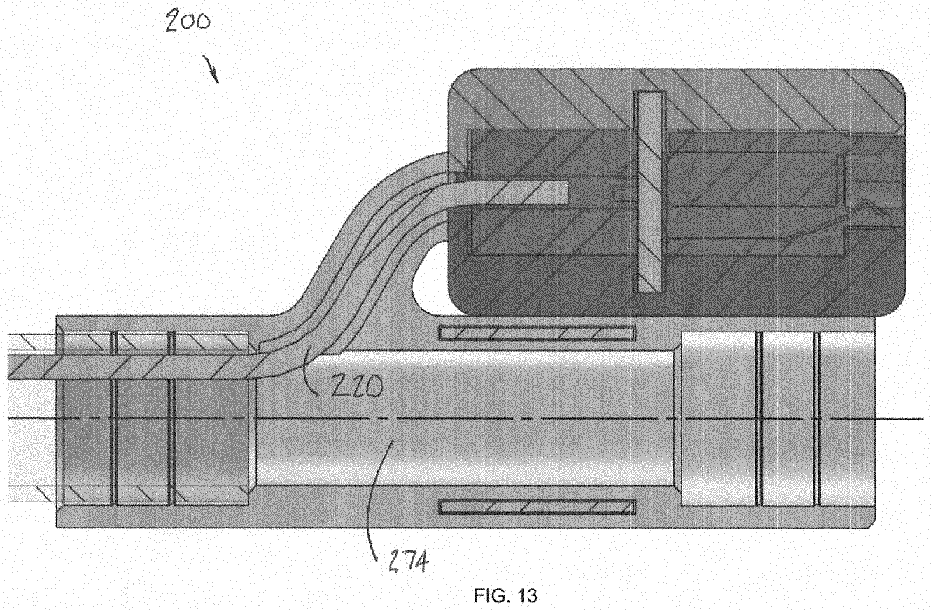

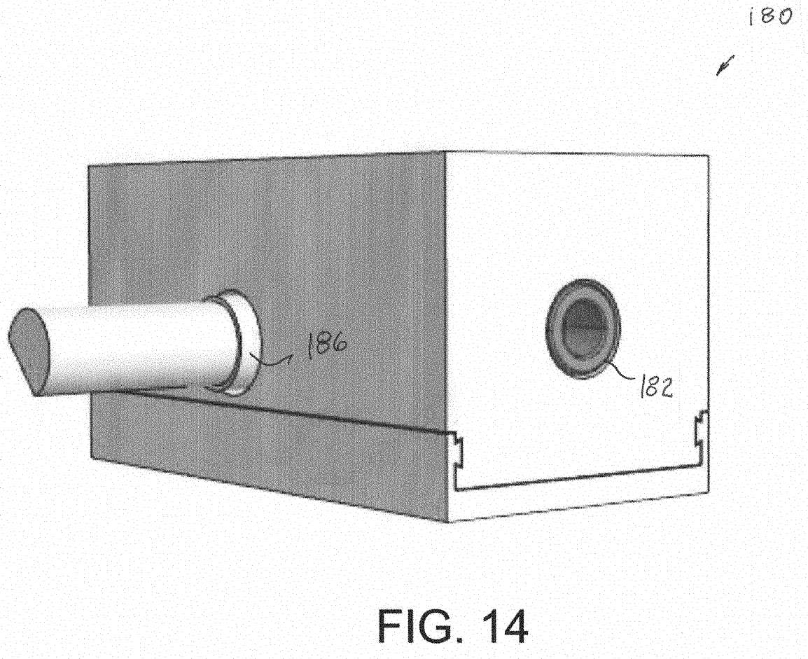

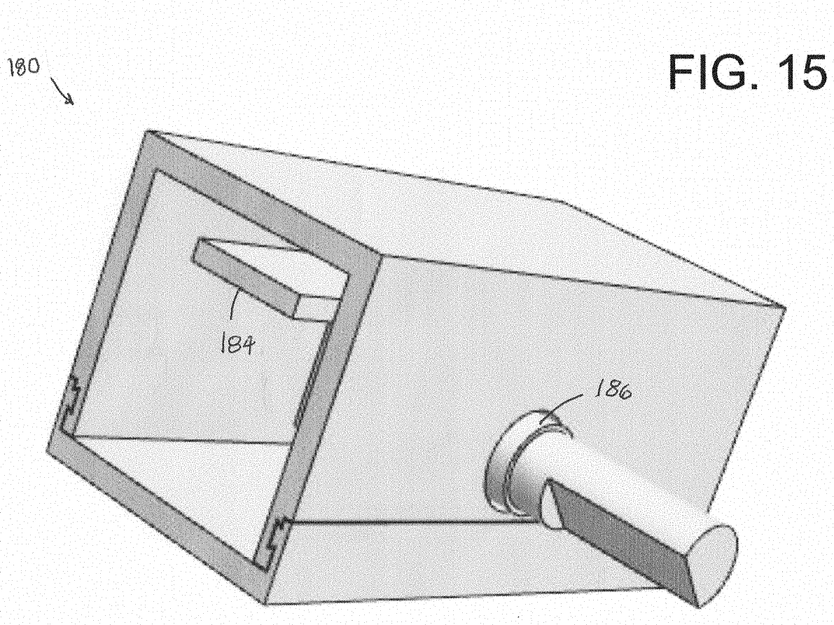

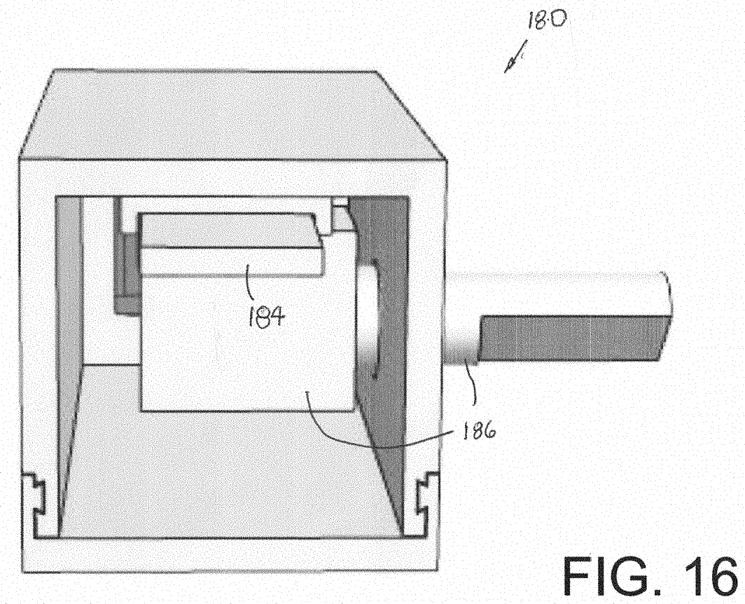

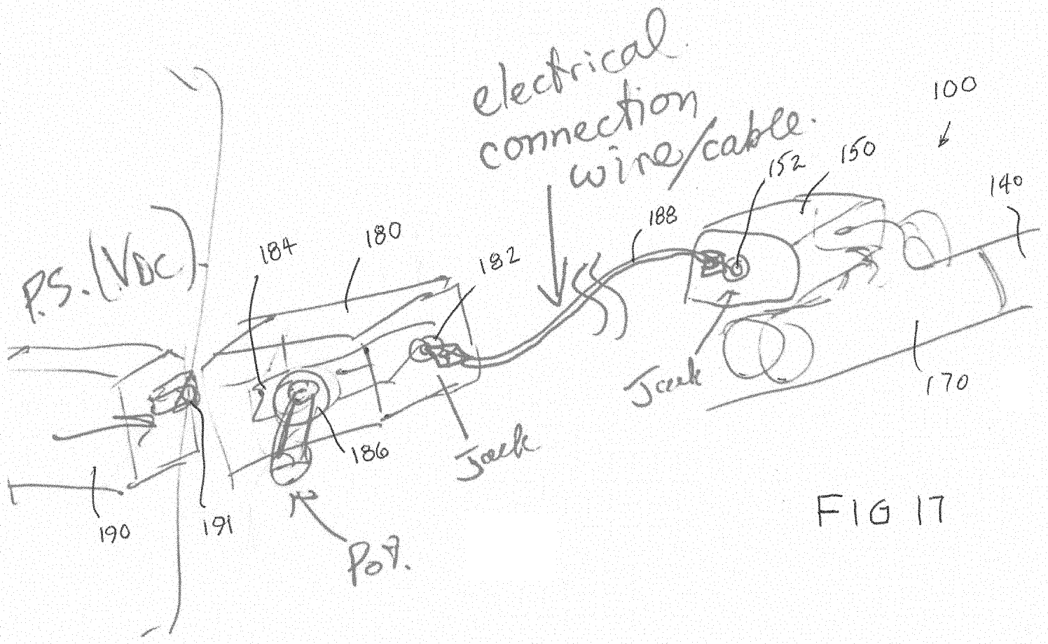

In the following specification, FIGS. 1 through 11 show the first illustrated embodiment of the work light according to the present invention, FIGS. 12 and 13 show the second illustrated embodiment of the work light according to the present invention, and FIGS. 14 through 17 show the power control assembly used in the first illustrated embodiment and the second illustrated embodiment of the work light according to the present invention.

In a first illustrated embodiment of the present invention, there is disclosed a novel work light, as indicated by the general reference numeral 100.

In the illustrated embodiment, but not necessarily, the work light 100 is used to present one or more LED lights 130 for illuminating and interior area. More specifically, the work light 100, as illustrated, comprises a dental work light and is used to illuminate a person's mouth. It has been found that there is a significant need in the field of dentistry for greater illumination in a patient's mouth when performing saliva suctioning, and just for general viewing.

In brief, the first illustrated embodiment work light 100 extends between a front end 101 and a back end 102, and comprises an elongate circuit board 120, a light emitting diode 130, an elongate protective cover 140, an electrical connector assembly 150, a main body 170, and a power control assembly 180.

The first illustrated embodiment work light 100 may comprise an elongate circuit board 120 having a length “LB” extending between a connection end 121c and a free end 121f, and a width “WB” extending between a left side 121 [symbol] and a right side 121r. As illustrated, the ratio of length “LB” to the width “WB” of the circuit board is 20:1 or greater in order to allow for a slim and narrow work light 100 that is readily grasped and manipulated in the hand of a dentist, dental assistant, or the like.

In order to provide sufficient light outwardly of the front end 101 and the surrounding area of the work light 100, a light emitting diode 130 is mounted on the elongate circuit board 120, preferable adjacent the free end of the elongate circuit board 120. More than one light emitting diode 130 can be mounted on the elongate circuit board 120, if desired, in order to achieve a required brightness. Also, the light emitted by the light emitting diode 130 or by a plurality of light emitting diodes, may be of any suitable light wavelength range. Typically a range of wavelengths that produces a bright white would be used for general illumination. Alternatively or additionally, a specific wavelength of light or narrow band of wavelengths of light such as a spectrum of blue light in the 400-500 nm range (with a peak wavelength of about 460 nm) could be used.

As illustrated, the portable work light 100 comprises the flexible circuit board 120 having a top surface 121t and a bottom surface 121b. These orientations are chosen for the sake of clarity and/or convenience and are not absolute as the portable work light 100 can be used in any orientation necessary.

In a first illustrated embodiment, the elongate circuit board 120 is flexible, and may be made from any suitable circuit board materials, such as an aluminium core printed circuit board wherein a very thin aluminium core has a very thin layer of electrically insulative material disposed on both faces of the aluminium core, and a copper trace on one or both outer faces of the insulative material. As taught, the flexible circuit board 120 comprises a thin and narrow aluminum core circuit board (0.060 thick and 0.080 wide) with the aluminum core 128 covered by an electrically insulative material 129i disposed on both surfaces 128a, 128b of the aluminium core 128, and a copper trace 129c on the electrically insulative material 129i that is over the top surface 128a of the aluminum core 128. As a result of its specific construction, the flexible circuit board 120 is somewhat flexible upwardly and downwardly, since the aluminium core 128, the electrically insulative material 129i, and the copper trace 129c can accommodate this direction of flexibility, but not side-to-side, since the aluminium core 128, the electrically insulative material 129i, and the copper trace 129c cannot accommodate this direction of flexibility.

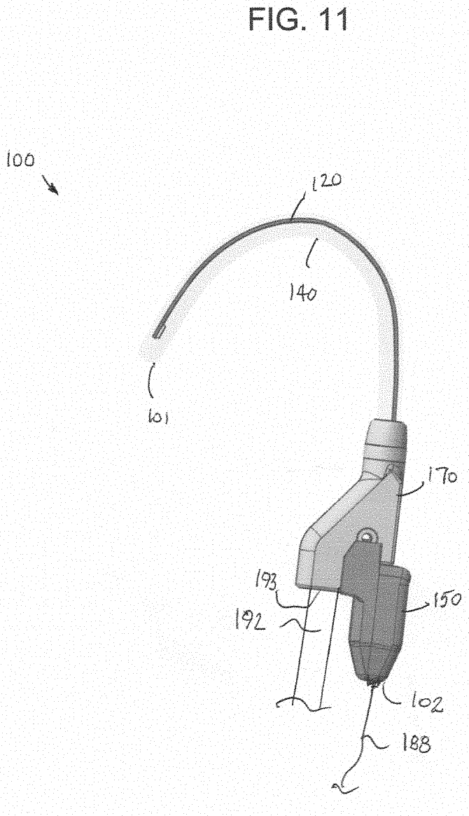

The purpose of using a flexible type of circuit board 120 is to be able to form the elongate circuit board 120 into a desired shape. As can be seen in FIG. 11, in the first illustrated embodiment, the elongate protective cover 140 and the flexible circuit board 120 therein have been bent to be able to hang on the mouth of a patient and suction saliva therefrom.

The elongate protective cover 140 extends between a mounting end 141 and a free end 142, between a left side 143 and a right side 144, and between a top 145 and a bottom 146. Further, the elongate protective cover 140 has a length “LC” defined as the distance between the mounting end 141 and the free end 142, a width “WC” defined as the distance between the left side 143 and the right side 144, and a height “HC” defined as the distance between the top 145 and a bottom 146. Further, because the elongate protective cover 140 is cylindrical with a circular cross-sectional area, the elongate protective cover 140 has a diameter “D”, which in this case is the same as the width “WC” and the height “HC”.

The elongate protective cover 140 generally surrounds at least most of the elongate circuit board 120, and in the first illustrated embodiment, the elongate protective cover 140 generally surrounds the elongate main portion 123 of the elongate circuit board 120, to thereby protect the surrounded portion of the elongate circuit board 120.

Also in the first illustrated embodiment, the elongate protective cover 140 comprises a tube 140 having a throughpassage 147 extending between the mounting end 141 and the free end 142. Further, the tube 140 comprises a suctioning tube, and as illustrated, a clear plastic flexible tube 140 that has a fluid ingress 148 at the free end 142 thereof and a fluid egress 149 at the mounting end 141 thereof. The back end portion of the elongate protective cover 140 is received into the fluid ingress 175 of the fluid-passing throughpassage 174 of the main body 170. The fluid egress 149 is for connecting the clear plastic flexible tube 140 in fluid transfer relation to the main body 170 and fluid transfer relation to the suctioning hose 192, to thereby pass saliva to the open front end of the suctioning hose 192 that is connected in fluid transfer relation to the suctioning apparatus 190. The suctioning tube 140 is thereby connectable at the mounting end 141 to a source of suction.

The fluid ingress 175 is shaped and dimensioned to receive the mounting end 141 of the elongate protective cover 140 in frictional engagement therein. As illustrated, the fluid-passing throughpassage 174 of the main body 170 is narrower in cross sectional area than the fluid ingress 175 and the fluid egress 176. Further, the fluid-passing throughpassage 174 of the main body 170 is the same cross sectional area as the throughpassage 147 of the elongate protective cover 140 and the internal throughpassage (not specifically shown) of the suctioning hose 192, in order to provide a substantially constant shape and cross-section of the overall flow passage for the suctioning of air.

The suctioning tube 140 is substantially non-collapsible, and perhaps even rigid, to thereby preclude collapse when fluids are suctioned through the throughpassage 147. Also, the suctioning hose 192 is substantially non-collapsible for the same reason.

As illustrated, the ratio of length to the width of the protective tube 140 is 20:1 or greater, the ratio of length to the height of the protective tube 140 is 20:1 or greater, and the ratio of length to the diameter of the protective tube 140 is 20:1 or greater, in order to allow for a slim and narrow work light 100 that is readily grasped and manipulated in the hand of a dentist, dental assistant, or the like.

In order to maximize the amount of light reaching the intended target, such as the mouth of a patient, or in other words to gain maximum benefit from the light being emitted from the light emitting diode 130, at least a portion of the elongate protective cover 140 at the light emitting diode 130 is made from a light-passing material. As illustrated, the light-passing material is transparent and the entire elongate protective cover 140 is made from a light-passing material, such as a suitable clear plastic material.

The work light 100 further comprises a main body 170 having a front end 171 and a back end 172. The protective cover 140 and the elongate circuit board 120 each extend forwardly from the main body 170. The main body 170 the main body 170 has a fluid ingress 175 and a fluid egress 176 interconnected in fluid communication by a fluid-passing throughpassage 174. The fluid ingress 175 is disposed at the front end 171 of the main body 170, and the fluid egress 176 is disposed at the back end 172 of the main body 170. The fluid ingress 175 is shaped and dimensioned to receive the mounting end 141 of the elongate protective cover 140 therein. The fluid egress 176 is shaped and dimensioned to receive the connection end 193 of the suctioning hose 192 therein.

A circuit board throughpassage 177 in the main body 170 extends from the inlet end 171 to the outlet end 172 of the main body 170 in order to facilitate the ready insertion of a back end portion 120b of the elongate circuit board 120 through the throughpassage 177 such that a projecting end portion 120p at the connection end 121c partially projects outwardly from the outlet end 172 of the main body 170.

The electrical connector assembly 150 comprises an outer housing 151 and an electrical connector circuit 150c securely mounted within and retained by the outer housing 151. The electrical connector circuit 150c comprises an input jack 152 and an edge connector 154 interconnected by suitable wires 156 to permit delivery of power from the input jack 152 to the edge connector 154. The electrical connector assembly 150 permits delivery of electrical power from a source of electrical power (not specifically shown) to the edge connector 150.

The elongate circuit board 120 is connected at the connection end 121c in electrically conductive relation to the circuit board connector 154, namely the edge connector 154, of the electrical connector assembly 150. In the first illustrated embodiment, the elongate circuit board 120 is physically connected at the connection end 121c to the circuit board connector 154 such that the connection end 121c of the circuit board 120 engages and is received in removable and replaceable relation with respect to the circuit board connector 154. The electrical connector assembly 150 is thereby operatively connected to the main body 170. Electrical power is thereby supplied to the elongate circuit board 120 and through the elongate circuit board 120 to the light emitting diode 130.

Reference will now be made to FIGS. 12 and 13, which show the second illustrated embodiment of the work light according to the present invention, as indicated by the general reference numeral 200. The second illustrated embodiment work light 200 is similar to the first illustrated embodiment work light 100, except that the fluid-conducting throughpassage 274 is straight and the flexible circuit board 220 is shaped into an “S”-shape.

Reference will now be made to FIGS. 14 through 17, which show a power control assembly 180 and source of DC power 190 used in both of the first illustrated embodiment work light 100 and the second illustrated embodiment work light 200 according to the present invention. The power control assembly 180 comprises a power input USB connector 184 that is electrically connected to a power output jack 182, or any other suitable type of electrical connector, through a suitable potentiometer or rheostat 186. The source of DC power 190, which suitably comprises a battery pack or the like 190, or any other suitable source of power, is electrically connected to USB connector 184 via a suitable cable such as a USB cable 192. The power output jack 182 is electrically connectable in power supplying relation to the input jack 152 of the electrical connector assembly 150 by a suitable electrical cable or wire 188, to deliver power thereto, and ultimately provide power to the light emitting diode 130 to illuminate the light emitting diode 130.

Other variations of the above principles will be apparent to those who are knowledgeable in the field of the invention, and such variations are considered to be within the scope of the present invention. Further, other modifications and alterations may be used in the design and manufacture of the impact mechanism of the present invention without departing from the spirit and scope of the accompanying claims.

1. A work light comprising:
an elongate circuit board having a length extending between a connection end and a free end and a width extending between a left side and a right side;
a light emitting diode mounted on said elongate circuit board; and
an elongate protective cover having a length extending between a mounting end and a free end, and generally surrounding at least most of said elongate circuit board.

2. A work light according to claim 1, wherein said light emitting diode is mounted on said elongate circuit board, adjacent said free end thereof.

3. A work light according to claim 1, wherein said elongate protective cover comprises a tube having a throughpassage extending between said connection end and said free end.

4. A work light according to claim 3, wherein said tube comprises a suctioning tube has a fluid ingress disposed at said free end thereof and a fluid egress disposed at said connection end thereof, and wherein said suctioning tube is connectable at said mounting end to a source of suction.

5. A work light according to claim 4, wherein said suctioning tube is substantially rigid to thereby preclude collapse when fluids are suctioned through said throughpassage.

6. A work light according to claim 1, wherein at least a portion of said elongate protective cover at said light emitting diode is made from a light-passing material.

7. A work light according to claim 1, wherein said elongate protective cover and said elongate circuit board are both flexible.

8. A work light according to claim 1, further comprising a main body 170 having a front end 171 and a back end 172, and wherein said protective cover and said elongate circuit board each extend forwardly from said main body 170.

9. A work light according to claim 8, wherein said main body 170 has a fluid ingress 175 and a fluid egress 176 interconnected in fluid communication by a fluid-passing throughpassage 174.

10. A work light according to claim 9, wherein said fluid ingress 175 is disposed at said front end 171 of said main body 170, and said fluid egress 176 is disposed at said back end 172 of said main body 170,

11. A work light according to claim 8, further comprising an electrical connector assembly 150 operatively connected to said main body 170.

12. A work light according to claim 8, wherein a back end portion of said elongate protective cover is received into said fluid ingress 175 of said fluid-passing throughpassage 174 of said main body at said fluid ingress 175.

13. A work light according to claim 12, wherein said elongate circuit board extends outwardly from the mounting end of said elongate protective cover and extends through a circuit board throughpassage in said main body.

14. A work light according to claim 13, wherein said elongate circuit board extends rearwardly from said main body and engages said electrical connector in removable and replaceable relation.

15. A work light comprising:
a circuit board;
a light emitting diode mounted on said circuit board;
a protective cover generally surrounding at least most of said circuit board;
wherein said protective cover comprises a suctioning tube having a fluid ingress and a fluid egress, and wherein said suctioning tube is connectable at said mounting end to a source of suction.

16. A work light according to claim 15, wherein said protective cover generally surrounds said light emitting diode.

17. A work light according to claim 15, wherein said circuit board has a length extending between a connection end and a free end and a width extending between a left side and a right side, and said protective cover has a length extending between a mounting end and a free end, said suctioning tube has a throughpassage extending between said mounting end and said free end, and wherein said fluid ingress is disposed at said free end thereof and said fluid egress is disposed at said connection end thereof.

18. A work light according to claim 17, wherein said circuit board is elongate and said protective cover is elongate.

19. A work light according to claim 15, wherein said light emitting diode is mounted on said elongate circuit board, adjacent said free end thereof.

20. A work light according to claim 15, wherein at least a portion of said elongate protective cover at said light emitting diode is made from a light-passing material.
